(19) United States
(12) Patent Application Publication
(10) Pub. No.: US 2009/0281197 A1
(43) Pub. Date: Nov. 12, 2009

(54) Acetic Acid/Thymol Compositions and Their Use in the Treatment of Onychomycosis

(76) Inventors: Paul Aaron Kinsinger, Washington, IL (US); John Preckshot, Peoria, IL (US)

Correspondence Address: WILLIAMS, MORGAN & AMERSON, 10333 RICHMOND, SUITE 1100, HOUSTON, TX, 77042, US

(21) Appl. No.: 12/358,474

(22) Filed: Jan. 23, 2009

Related U.S. Application Data
(60) Provisional application No. 61/023,605, filed on Jan. 25, 2008.

(51) Int. Cl. A61K 31/05; A61K 47/12; A61P 31/10

(52) U.S. Cl. 514/729; 514/784

(57) ABSTRACT

A composition comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel; wherein the saturated carboxylic acid, the antifungal compound, and the skin-penetrating gel together comprise 100 weight parts. In a particular example of the composition, the saturated carboxylic acid having from 2 to about 20 carbon atoms is acetic acid; and the antifungal compound is thymol. A method of treating onychomycosis in a patient, comprising applying the composition. The method provides a safe, effective treatment of onychomycosis.

This application claims priority from U.S. provisional patent application Ser. No. 61/023,605, filed on Jan. 25, 2008, which is incorporated herein by reference.

### BACKGROUND OF THE INVENTION

The present invention relates to the field of medical treatments for infections of the nails.

Onychomycosis is used herein to refer to any fungal infection of a nail. Onychomycosis typically manifests as a thickening, discoloration, or both of the infected nail, which may lead to pain, discomfort from wearing shoes or trimming the infected nail, a subjective feeling of embarrassment (especially if the nail is a fingernail), or even complete loss of the nail. (The word “or” is used throughout this document in the inclusive, “and/or” sense, unless a particular use is explicitly stated to be exclusive). Although onychomycosis can strike a person of any age or either sex, approximately 10% of Americans over age 65 are affected with oncyhomycosis.

Because the infection may reside under the nail plate, in a warm, moist environment amenable to fungi, existing treatments are of dubious efficacy. Regarding topical remedies, one home remedy involves soaking the affected nail in a mixture of white vinegar and water for five minutes daily. However, the only evidence for this home remedy is merely anecdotal. Currently in the United States, two topical medications approved for over-the-counter use are available: clotriamazole and tolnaftate. These active ingredients are found in about 80% of over-the-counter onychomycosis medications. Scher, et al., Dial. Dermatol. Comment. (November 2002) 51(2) mention that 40% urea may have a keratolytic (dead-skin removing) effect. American Family Physician's practical therapeutic section (vol. 63, no. 4, Feb. 15, 2001) mentions topical applications only in conjunction with concomitant tinea pedis. Archives of Dermatol. (2002) 138:811-816 reviewed research reports and concluded that onychomycosis was unlikely to improve with only topical treatment, though the article also suggested that most of the published work on the topic was funded by the pharmaceutical industrial, which presumably has an oral product basis). Also, Dr. Michael Lehrer, Dept. of Dermatology, University of Pennsylvania, wrote in a medical encyclopedia article dated October 2006 that over-the-counter creams and ointments do not help treat onychomycosis. In summary, current medical literature in family medicine, internal medicine, and podiatry does not support topical treatment of onychomycosis.

Oral medications for onychomycosis are reported by their package inserts to be 70-80% effective. Published reviews generally report lower efficacy rates, e.g., oral terbinafine achieved a disease-free nail in 35-50% of patients, whereas oral itraconazole's result was 25-40% (Arch. Dermatol. 134(12) (December 1998)). Also, no disease recurrence data was given for either product. American Family Physician's practical therapeutic section (vol. 63, no. 4, Feb. 15, 2001) mentions oral griseofulvin in children has some level of toxicity. In general, when treating onychomycosis with the oral medications discussed above, both monthly liver function studies and frequent visits to a physician's office for supervision are recommended. There also exists some risk of interactions with other medications. Also, use of the oral medications discussed above during pregnancy may be discouraged.

Therefore, the need exists for safe, effective treatments of onychomycosis.

### SUMMARY OF THE INVENTION

In one embodiment, the present invention relates to a composition, comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel.

In one embodiment, the present invention relates to a method of treating onychomycosis in a patient, comprising applying a composition comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel.

The method provides a safe, effective treatment of onychomycosis.

### DESCRIPTION OF ILLUSTRATIVE EMBODIMENTS

In one embodiment, the present invention relates to a composition, comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel; wherein the saturated carboxylic acid, the antifungal compound, and the skin-penetrating gel together comprise 100 weight parts.

Saturated carboxylic acids having from 2 to about 20 carbon atoms are known in the art. Particular examples include acetic acid, propionic acid, butyric acid, valeric acid, caproic acid, enanthic acid, caprylic acid, pelargonic acid, capric acid, lauric acid, palmitic acid, and stearic acid. “Acid” is used herein to refer to both the protonated form and the deprotonated form of the compound.

“Antifungal compound” refers to a compound known to the person of ordinary skill in the art to be reputed to kill or retard the growth or reproduction of one or more fungi. An exemplary antifungal compound is thymol.

A “skin-penetrating gel” refers to any gel known to the person of ordinary skill in the art of pharmaceutical compounding to be useful as a carrier for topical application of an active compound to the skin of a vertebrate, wherein the active compound has a greater rate of penetration of the skin than it has when applied without being carried by the skin-penetrating gel. The continuous phase of the gel itself need not have a greater rate of penetration of the skin for the gel to be “skin-penetrating.” An exemplary skin-penetrating gel is Roentsch gel, which will be discussed in more detail below. Xanthan gum is not a skin-penetrating gel as the term is used herein.

Exemplary skin-penetrating gel ingredients are discussed by Percutaneous Penetration Enhancers, Eric W. Smith, Howard I. Maibach, Edition: 2, illustrated, CRC Press, 2006, ISBN 0849321522, 9780849321528, which is hereby incorporated herein by reference.

In one embodiment, the composition comprises from about 1 weight part to about 10 weight parts acetic acid; from about 1 weight parts to about 10 weight parts thymol; and from about 80 weight parts to about 98 weight parts skin-penetrating gel; wherein the acetic acid, the thymol, and the skin-penetrating gel together comprise 100 weight parts.

“Acetic acid” is used herein to refer to both the protonated form (having the molecular formula CH₃COOH) and the deprotonated form (CH₃COO⁻). In aqueous solutions, the pKₐ of acetic acid is about 4.76 at 25° C. Alternate names for acetic acid include ethanoic acid, acetyl hydroxide, hydrogen acetate, ethylic acid, and methanecarboxylic acid. The structural formula of acetic acid is:

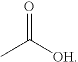

Thymol is also known as isopropylmethylphenol, isopropyl-m-cresol, or hydroxyl cymene. It has the molecular formula C₁₀H₁₄O and the structural formula:

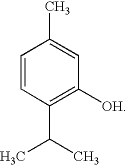

Roentsch gel is a skin-penetrating gel known to the person of ordinary skill in the pharmaceutical compounding art. Roentsch gel was originally formulated by E. George Roentsch, a compounding pharmacist having a place of business at 35 Main St., Keene, N.H. 03431.

In one embodiment, a roughly 200 ml volume of skin-penetrating gel contains 126 ml propylene glycol, 68 ml ethoxy diglycol, 4 g hydroxypropyl cellulose NF 1500 cps, 0.16 g menthol crystals USP, 0.10 g butylated hydroxytoluene (BHT), and 0.5 g decylmethyl sulfoxide.

In one embodiment, the composition is formed by dissolving thymol crystals in acetic acid, bringing to final volume with the skin-penetrating gel, and lavigating the mixture until homogeneous.

In another embodiment, the composition comprises propylene glycol 60.35 volume parts, Transcutol® CG (diethylene glycol monoethyl ether) 33.00 volume parts, Methocel 4AC (methyl cellulose) 4.00 weight parts, menthol 0.10 weight parts, BHT 0.05 weight parts, ARLASOW DMI 0.25 weight parts, thymol 0.25 weight parts, and acetic acid 2.00 weight parts.

Acetic acid is, along with water, a primary ingredient of vinegar, which is clearly acceptable for human consumption in reasonable doses. Thymol takes its name from the thyme plant, an herb commonly used for human consumption. Skin-penetrating gels, such as Roentsch gel, are known to be inert. Therefore, the combination of these three materials is expected to be safer than known oral treatments for onychomycosis.

The composition may comprise one or more other materials. In one embodiment, the composition comprises one or more other materials known in the art to be useful in compositions intended for topical administration.

In one embodiment, the composition further comprises water. In further embodiment, the composition can further comprise one or more solutes, such as salts, acids, bases, or mixtures thereof, among others. The composition can also comprise one or more of a surfactant or an emulsifier.

In one embodiment, the composition further comprises one or more polar organic solvents. “Polar” has its standard meaning in the chemical arts of describing a molecule that has a permanent electric dipole. A polar molecule can but need not have one or more positive, negative, or both charges. Examples of polar organic solvents include, but are not limited to, methanol, ethanol, formate, acrylate, or mixtures thereof, among others. The composition can further comprise one or more solutes, such as salts, among others. The composition can also comprise one or more of a surfactant or an emulsifier.

In one embodiment, the composition further comprises one or more polar organic solvents. “Polar” has its standard meaning in the chemical arts of describing a molecule that does not have a permanent electric dipole. Examples of apolar organic solvents include, but are not limited to, hexane, cyclohexane, octane, toluene, benzene, or mixtures thereof, among others. The composition can further comprise one or more solutes, such as apolar molecules, among others. The composition can also comprise one or more of a surfactant or an emulsifier.

In one embodiment, the composition further comprises a mixture of water and other solvents. In one embodiment, the composition can comprise one or more of dimethicone, water, urea, mineral oil, sodium lactate, polyglyceryl-3 diisostearate, ceresin, glycerin, octyldodecanol, polyglyceryl-2 dipolyhydroxystearate, isopropyl stearate, panthenol, magnesium sulfate, bisabolol, lactic acid, lanolin alcohol, or benzyl alcohol, among others.

In one embodiment, the composition has a creamy consistency suitable for packaging in a squeezable plastic container. In one embodiment, the composition has a lotion consistency suitable for packaging in a squeezable plastic container. In one embodiment, the composition has an ointment-like consistency suitable for packaging in a squeezable plastic container. In one embodiment, the composition has a liquid consistency suitable for packaging in a non-squeezable container. A non-squeezable container can be fabricated from one or more of plastic, glass, metal, ceramic, or other compounds. A non-squeezable container can be fabricated with a flow-type cap or a pump-type dispenser.

Other materials that may be included in the composition will be apparent to the skilled artisan having the benefit of the present disclosure.

In one embodiment, the composition is pharmaceutically-acceptable. By “pharmaceutically-acceptable” is meant that the composition is suitable for use in medicaments intended for topical administration to a mammal. Parameters which may considered to determine the pharmaceutical acceptability of a composition can include, but are not limited to, the toxicity of components of the composition, the interaction between components of the composition, the approval by a regulatory body of the composition or its components for use in medicaments, or two or more of the foregoing, among others.

Acetic acid, thymol, and skin-penetrating gels, such as Roentsch gel, all either are or are expected to be pharmaceutically acceptable in the United States and other countries.

In addition to the acetic acid, thymol, and skin-penetrating gel, and any further components described above, the composition can also further comprise other compounds, such as preservatives, adjuvants, excipients, binders, diluents, surfactants, or other agents capable of treating one or more diseases, or mixtures thereof, among others.

In one embodiment, the composition comprises from about 2 weight parts to about 8 weight parts acetic acid; from about 2 weight parts to about 6 weight parts thymol; and from about 86 weight parts to about 96 weight parts skin-penetrating gel.

In a further embodiment, the composition comprises from about 4 weight parts to about 6 weight parts acetic acid; about 4 weight parts thymol; and from about 90 weight parts to about 92 weight parts skin-penetrating gel.

In one embodiment, the present invention relates to a method of treating onychomycosis in a patient, comprising:

applying a composition comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel; wherein the saturated carboxylic acid, the antifungal compound, and the skin-penetrating gel together comprise 100 weight parts, to a nail of the patient.

The composition can be as described above. In one embodiment, the composition comprises from about 1 weight part to about 10 weight parts acetic acid; from about 1 weight parts to about 10 weight parts thymol; and from about 80 weight parts to about 98 weight parts skin-penetrating gel.

Onychomycosis is used herein to refer to any fungal infection of a nail. A particular case of onychomycosis may be caused by one or more microorganisms of the genera Tricophyton, Epidermophyton, Microsporum, Candida, Neoscytalidium, Scopulariopsis, or Aspergillus, among others. The one or more microorganisms of the genus Tricophyton may be of the species T. rubrum, T. interdigitale, T. violaceum, T. tonsurans, T. soudanense, or T. verrucosum. However, a particular case of onychomycosis may be caused by a microorganism not set forth above. Onychomycosis may also be referred to as tinea unguium, depending on the causative microorganism(s).

A particular case of onychomycosis may manifest as one or more of distal subungual onychomycosis (an infection of the nail bed and the underside of the nail plate), superficial onychomycosis (an infection of the outer surface of the nail plate), proximal subungual onychomycosis (an infection of newly formed nail plate through the proximal nail fold), candidal onychomycosis, or total dystrophic onychomycosis (loss of the nail plate; a possible end result of any of the above).

The nail of the patient may be a fingernail or a toenail. It is possible for a patient to have infections of two or more nails.

“Nail” is used to refer to any hard structure predominantly comprising keratin located at the end of a finger, toe, or structure overlaying a distal phalanx of a vertebrate. In dogs, cats, and birds, the nail may be referred to as a claw; in horses, the nail may be referred to as a hoof. In light of this, the patient may be other than a human being, e.g., a dog, a cat, a bird, or a horse, among other vertebrates having economic or esthetic utility to human beings.

Treating is used herein to refer to a reduction in the severity, a delay of the progression, or both of a particular case of onychomycosis. A reduction in the severity can be shown by a reduction in the surface area of the nail showing onychomycosal symptoms such as thickening, yellowing, or cloudiness of the nail plate, or separation of the nail plate from the nail bed; easier trimming of the nail; a reduction in pain when shoes are worn over toenails; or a subjective assessment by the patient, a physician, or another observer that the nail appears healthier. A delay in progression can be determining the infection's progress when treating begins, following the patient's symptoms over time until a desired time point is reached, and comparing the patient's symptoms at the desired time point against a database of observations of control infections taking over one or more time points. A physician can readily assemble or gain access to such a database. For example, the delay in progression can be shown as a decreased percentage of lost or removed nails after a desired time duration for a treated group relative to an untreated control group.

The dosage amount, the dosage frequency, and the duration of the treatment regimen can vary according to the judgment of a medical practitioner. In one embodiment, the dosage amount is from about 0.05 cc to about 0.5 cc, i.e., applying comprises applying from about 0.05 cc to about 0.5 cc of the composition to the nail of the patient. An exemplary dosage is about 0.15 cc, or roughly the volume of a pencil eraser. The dosage may be applied to the surface of the nail plate, to the distal nail fold, to the proximal nail fold, to either or both lateral nail folds, or two or more thereof.

In one embodiment, the dosage frequency is from once per day to about four times per day, i.e., applying is performed from one time per day to about four times per day.

The treatment regimen can be continued until the patient's symptoms partially improve or completely improve, i.e., until no symptoms of onychomycosis are seen. The treatment regimen may last for three months, six months, one year, or even more, as needed. In certain embodiments, however, the treatment regimen may last for less than three months.

In addition to applying the composition, in one embodiment, the invention further comprises covering the nail of the patient. Covering can be effected by a bandage, a gauze, a poultice, or the like. In one covering is for a duration from about 30 min to about eight hours.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

Approximately 10% of Americans over age 65 are affected with oncyhomycosis. One home remedy involves soaking the affected nail in a mixture of white vinegar and water for five minutes daily. However, the evidence supporting the use of vinegar/water mixtures is merely anecdotal. Oral medications for onychomycosis are reported by their package inserts to be 70-80% effective. However, the oral medications require both monthly liver function studies and frequent visits to a physician's office for supervision, run the risk of interactions with other medications, and are not advised during pregnancy. Currently in the United States, two topical medications approved for over-the-counter use are available: clotriamazole and tolnaftate. These active ingredients are found in about 80% of over-the-counter onychomycosis medications. However, current medical literature in family medicine, internal medicine, and podiatry does not support topical treatment of onychomycosis.

Our study aims to determine the efficacy of a topical formulation of acetic acid and thymol in an inert, skin-penetrating holding agent versus topical formulations of clotriamazole or tolnaftate. All formulations will be administered and followed by covering the nail with a bandage for at least two hours. The occlusive bandage and inert holding agent, without active ingredients, will also be tested.

A new toenail scale will be used to obtain objective evidence along with photographs at the initial evaluation, three months, and six months. The daily treatment period will be about three months (12 weeks). Compliance rates will also be recorded. The two study sites will include a family medicine residency clinic and a podiatry clinic. Efficacy of the four treatment arms (acetic acid/thymol, clotriamazole, tolnaftate, or bandage alone) will not be determined by spore cultures of the nail. Photographs, as stated above, will be used to determine whether visually the onychomycosis is cleared at three months and six months (three months after discontinuing daily treatment). The six month study will be double blinded and compliance, as well as any side effects of the 12-week daily treatment, will be recorded.

Specifically, the formulations that will be tested are as follows:

| Formulation | Composition and Treatment Regimen |
|---|---|
| I | 4% acetic acid, 4% thymol, balance Roentsch gel |
| | Approx. 0.15 cc, once daily, followed by 2 hr bandage, |
| | 12 weeks |
| II | 1% clotriamazole, balance xanthan gum |
| | Label dose, once daily, followed by 2 hr bandage, 12 weeks |
| III | 1% tolnaftate, balance xanthan gum |
| | Label dose, once daily, followed by 2 hr bandage, 12 weeks |
| IV | 100% Roentsch gel (no acetic acid, thymol, clotriamazole, |
| | or tolnaftate) Approx. 0.15 cc, once daily, followed by 2 hr |
| | bandage, 12 weeks |

Formulations II and III are comparable to known over-the-counter antifungal medicaments comprising clotriamazole or tolnaftate and xanthan gum as an inert holding agent.

The study's objectives are:

1. Determine if Formulation I, administered as described above, is useful in the treatment of onychomycosis, as shown by visual improvement or disappearance of infection at three and six months and recurrence rate at six months.

2. Determine if Formulation II, administered as described above, is useful in the treatment of onychomycosis.

3. Determine if Formulation III, administered as described above, is useful in the treatment of onychomycosis.

4. Determine if Formulation I is superior or equal to Formulation II, Formulation III, or both, all administered as described above.

5. Determine if Formulation IV, administered as described above, is useful in the treatment of onychomycosis.

6. Monitor adverse events of Formulations I-IV.

7. Determine compliance rates needed for successful treatment.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition, comprising:
from about 1 weight part to about 10 weight parts a saturated carboxylic acid having from 2 to about 20 carbon atoms;
from about 0.1 weight parts to about 10 weight parts an antifungal compound; and
from about 80 weight parts to about 99 weight parts skin-penetrating gel;
wherein the saturated carboxylic acid, the antifungal compound, and the skin-penetrating gel together comprise 100 weight parts.

2. The composition of claim 1, wherein the saturated carboxylic acid having from 2 to about 20 carbon atoms is acetic acid; and the antifungal compound is thymol.

3. The composition of claim 2, comprising from about 2 weight parts to about 8 weight parts acetic acid; from about 2 weight parts to about 6 weight parts thymol; and from about 86 weight parts to about 96 weight parts skin-penetrating gel.

4. The composition of claim 3, comprising from about 4 weight parts to about 6 weight parts acetic acid; about 4 weight parts thymol; and from about 90 weight parts to about 92 weight parts skin-penetrating gel.

5. A method of treating onychomycosis in a patient, comprising:
applying a composition comprising from about 1 weight part to about 10 weight parts a saturated carboxylic acid having from 2 to about 20 carbon atoms; from about 0.1 weight parts to about 10 weight parts an antifungal compound; and from about 80 weight parts to about 99 weight parts skin-penetrating gel; wherein the saturated carboxylic acid, the antifungal compound, and the skin-penetrating gel together comprise 100 weight parts, to a nail of the patient.

6. The method of claim 5, wherein the saturated carboxylic acid having from 2 to about 20 carbon atoms is acetic acid; and the antifungal compound is thymol.

7. The method of claim 6, wherein applying comprises applying from about 0.05 cc to about 0.5 cc of the composition to the nail of the patient.

8. The method of claim 6, wherein applying is performed from one time per day to about four times per day.

9. The method of claim 6, further comprising covering the nail of the patient.

10. The method of claim 9, wherein covering is for a duration from about 30 min to about eight hours.

11. The method of claim 6, wherein the composition comprises from about 2 weight parts to about 8 weight parts acetic acid; from about 2 weight parts to about 6 weight parts thymol; and from about 86 weight parts to about 96 weight parts skin-penetrating gel.

12. The method of claim 11, wherein the composition comprises from about 4 weight parts to about 6 weight parts acetic acid; about 4 weight parts thymol; and from about 90 weight parts to about 92 weight parts skin-penetrating gel.
